(19) United States
(12) Patent Application Publication
(10) Pub. No.: US 2016/0324798 A1
(43) Pub. Date: Nov. 10, 2016

(54) POLYMER-BASED COMPOSITIONS FOR EXTENDED RELEASE OF PROTEINS

(71) Applicant: HERON THERAPEUTICS, INC., Redwood City, CA (US)

(72) Inventors: Han Han, Belmont, CA (US); Thomas B. Ottoboni, Belmont, CA (US); Laura Lerner, Foster City, CA (US)

(21) Appl. No.: 15/108,986

(22) PCT Filed: Dec. 30, 2014
(86) PCT No.: PCT/US14/72855

§ 371 Date: Jun. 29, 2016

Related U.S. Application Data
(60) Provisional application No. 61/922,739, filed on Dec. 31, 2013.

(51) Int. Cl. A61K 9/70; A61K 38/38; C07K 16/00; A61K 47/34

(52) U.S. Cl. CPC A61K 9/7007; A61K 47/34; A61K 38/38; C07K 16/00

(57) ABSTRACT

The present application is directed to poly(orthoester)-based formulations that are effective for the sustained delivery of one or more therapeutic proteins. The formulations additionally maintain the stability and bioactivity (i.e., significantly minimize the degradation and/or aggregation) of the protein contained in the poly(orthoester) matrix during preparation, storage and release.

### CROSS-REFERENCE TO RELATED APPLICATIONS

This application claims the benefit of priority to U.S. Provisional Patent Application No. 61/922,739, filed Dec. 31, 2013, the content of which is incorporated herein by reference in its entirety.

### FIELD

The disclosure relates generally to poly(orthoester)-based formulations and systems for the prolonged release of therapeutic proteins, among other features.

### BACKGROUND

The development of therapeutic proteins has revolutionized healthcare by providing unique treatments for numerous diseases and disorders. Examples of therapeutic protein drugs that have been approved for human use include human growth hormone, follicle stimulating hormone, Factor VIII, erythropoietin, granulocyte stimulating-colony factor, and interferon-beta, to name a few. A major drawback of administering protein therapeutics is the need for frequent and repeated administration, often daily or even multiple times a day, typically by intravenous infusion or subcutaneous injection, to achieve therapeutically effective levels of protein in the bloodstream. In an effort to improve patient compliance and convenience, and reduce the frequency of administration, the development of sustained release dosage forms of therapeutic proteins has been pursued.

One such approach is microencapsulation using biodegradable polymers such as poly(lactic acid) (PLA), poly(glycolic acid) (PGA), and poly(D,L-lactic-co-glycolic) acids (PLGA), which degrade via bulk erosion (Giteau, A., et al., International Journal of Pharmaceutics, 2008, 350 (1-2):14-26). Poly(D,L-lactic-co-glycolic) acids (PLGA) have been widely studied and used as biodegradable controlled release drug carriers. PLGAs are extremely attractive as drug carriers for pharmaceutical products and/or devices since they are approved by the United States Food and Drug Administration (FDA) (Makadia, H K; Siegel S J., Polymers, 2011, 3, 1377-1397). Several small molecule-PLGA based products are currently on the market including PLGA-encapsulated forms of leuprolide, octreolide and gosarelin. A sustained delivery form of human growth hormone encapsulated in PLGA microspheres was approved and successfully launched as Nutropin Depot®, however, the product was later withdrawn from the market.

The successful development of sustained delivery forms of therapeutic proteins continues to present a challenge. Various proteins have been reported to be unstable upon encapsulation in PLGA-delivery systems, generally due to peptide bond fragmentation, aggregation, and/or loss of bioactivity (Schwendeman, Steven P., Critical Reviews™ in Therapeutic Drug Carrier Systems, 19 (1), (2002); Estey, T.; Kang, J.; Schwendeman, S. P.; Carpenter, J. F., Journal of Pharmaceutical Sciences 2006, 95, (7), 1626-39). The degradation and aggregation of PLGA-encapsulated proteins is mainly attributed to proteins being exposed to an acidic environment that is caused by the rapid accumulation of trapped acidic PLGA degradation products during release.

Drug release from PLGA microspheres is generally due to drug diffusion through water-filled networks of pores and channels, coupled with bulk erosion of the microspheres by hydrolysis (Giteau, A., et al., International Journal of Pharmaceutics, 2008, 350 (1-2):14-26). While this model tends to work well for small hydrophobic drug molecules, this is not necessarily the case for therapeutic proteins in which a high initial burst followed by very slow or no release of protein is often observed (Giteau, A., ibid).

Thus, it would be highly advantageous to provide a delivery system which can provide sustained release of protein over time, while also maintaining the stability of the protein during formulation, in encapsulated form, and upon release in vivo.

### SUMMARY

In a first aspect, provided herein is a composition that is effective to provide extended release of a protein comprised within the composition. The composition comprises a poly(orthoester) combined with a therapeutic protein, where the poly(ortho-ester) comprises less than 5 mole percent of α-hydroxy acid-containing, i.e., latent acid, subunits, and has a glass transition temperature (Tg), of greater than about −10° C.

In some cases, the composition may possess a Tg of greater than about 0° C., or even greater than about 10° C.

In some embodiments related to the foregoing, the poly(orthoester) comprises from 0.01 to 4.9 mole percent α-hydroxy acid-containing (i.e., latent acid) subunits selected from subunits comprising lactide, glycolide or combinations thereof. In yet some other embodiments, the poly(orthoester) comprises no a-hydroxy acid-containing (i.e., latent acid) subunits.

In some embodiments, the poly(orthoester) possesses the following structure:

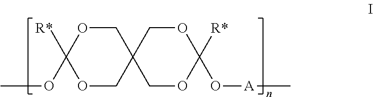

where:

R* is a C₁₋₄ alkyl;

n is an integer ranging from 5-400;

and A in each subunit is R¹, R², or R³, where

R¹ is:

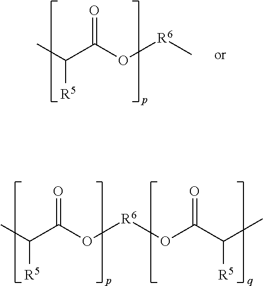

where:

p and q are each independently integers that vary from between about 1 to 20,

each R⁵ is independently hydrogen or C₁₋₄ alkyl; and

R⁶ is:

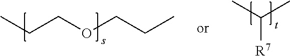

where s is an integer from 0 to 30;

t is an integer from 2 to 200; and

R⁷ is hydrogen or C₁₋₄ alkyl;

R² is:

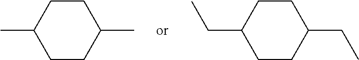

R³ is:

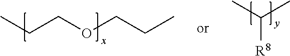

where:

x is an integer of 0 to 100;

y is an integer of 2 to 40; and

R⁸ is hydrogen or C₁₋₄ alkyl.

In some preferred embodiments related to formula I, A is R¹ or R³, where R¹ is

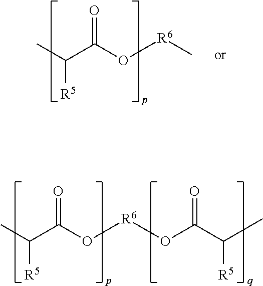

where p and q are each independently integers that vary from between about 1 and 20, where the average number of p or the average number of the sum of p and q (p+q) is between about 1 and 7 in at least a portion of the monomeric units of the polymer;

x and s are each independently an integer ranging from 0 to 30 (i.e., selected from 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30); and

t and y are each independently an integer ranging from 2 to 40 (i.e., selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40).

In yet some further preferred embodiments related to structure I, R⁵ is H or methyl.

In yet some additional embodiments, x is selected from 0, 1, 2, 3, 4, and 5.

In yet some further embodiments, the therapeutic protein is suitable for ophthalmic use. In an embodiment related to the foregoing, the therapeutic protein is aflibercept or ranibizumab.

In some preferred embodiments, the poly(orthoester) is a solid at room temperature.

In yet some further embodiments, the composition is in the form of microparticles such as microspheres or cylindrical rods.

In yet some additional embodiments, the composition and/or microparticles have a therapeutic protein loading of 75 weight percent or less.

In yet some other embodiments, the composition and/or microparticles comprise up to about 75 weight percent of one or more excipients.

Compositions such as those set forth above are effective to provide sustained release of the therapeutic protein over an extended period of time. For instance, in some particular embodiments, the composition is effective to provide sustained release of the therapeutic protein over a period of at least 10 days when evaluated in vitro in phosphate buffered saline at 37° C.

As an additional feature, in some embodiments, compositions such as those set forth above are effective to impart enhanced stability to the therapeutic protein contained therein. In some embodiments, a composition such as provided herein is further characterized by a degree of degradation of the therapeutic protein of no more than 25% when evaluated in vitro in phosphate buffered saline at 37° C. at day 7.

In some particular embodiments of a composition as provided herein, the combined mole percentage of

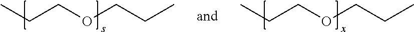

in the poly(orthoester) is less than 15.

In yet some further embodiments, a composition as provided herein comprises a poly(orthoester) prepared by reacting at least (i) from 30 to 60 mole percent 3,9-di(ethylidene)-2,4,8,10-tetraoxaspiro[5.5]undecane, (ii) from 10 to 50 mole total mole percent of two or more organic diols having a hydrocarbyl core of from 2 to 40 carbon atoms, and optionally having 1 to 3 elements of unsaturation, and (iii) less than 5 mole percent of an α-hydroxy-acid containing polymeric reactant under conditions effective to provide a poly(orthoester) polymer having a Tg of greater than 0° C. and that is a solid at room temperature.

In some embodiments related to the foregoing, the two or more organic diols have a hydrocarbyl core of from 2 to 20 carbon atoms.

In yet some additional embodiments, the poly(orthoester) is prepared by further including in the reacting step, a hydroxyl or α-hydroxy-acid terminated ethylene-glycol having from 2 to 30 subunits. In some even more specific embodiments, the hydroxyl or α-hydroxy-acid terminated ethylene glycol is a triethylene glycol.

In a second aspect, provided herein is a plurality of microparticles comprising a solid therapeutic protein contained within a poly(orthoester) matrix, wherein the poly(ortho ester) comprises less than 5 mole percent of α-hydroxy acid-containing subunits, and has a glass transition temperature (Tg), of greater than about −10° C.

In some embodiments related to the second aspect, the plurality of microparticles possess sizes ranging from about 4 microns to about 80 microns.

In some further embodiments related to the second aspect, provided is a plurality of microparticles as previously described, where the plurality of microparticles is effective to provide extended release of the therapeutic protein over a period of time that is extended by at least two-fold when compared to the release of the same therapeutic protein from a plurality of PLGA (50:50) microspheres when evaluated in vitro in phosphate buffered saline at 37° C. at day 7.

In yet some additional embodiments related to the second aspect, and any one or more of its related embodiments, provided is a plurality of microspheres containing a therapeutic protein, wherein the therapeutic protein is characterized by a degree of degradation of no more than 25% when evaluated in vitro in phosphate buffered saline at 37° C. at day 7.

In a third aspect, provided herein is a method for enhancing the stability of a therapeutic protein upon encapsulation in a polymeric matrix. The method comprises combining the therapeutic protein in a poly(orthoester) matrix, wherein the poly(orthoester) comprises less than 5 mole percent of α-hydroxy acid-containing subunits, and has a glass transition temperature (Tg), of greater than about −10° C.

In yet a fourth aspect, provided herein is a method of treating a mammalian subject for a condition that is treatable by administration of a therapeutic protein, the method comprising administering to the subject a therapeutically effective amount of a composition or the plurality of microparticles having the features set forth above.

Additional embodiments of the compositions, plurality of microparticles, methods, uses and the like will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description, particularly when considered in conjunction with the accompanying examples and drawings.

### BRIEF DESCRIPTION OF THE FIGURES

FIG. 1 is a plot illustrating the cumulative in-vitro release of the antibody, IgG, from various poly(ortho ester) microsphere formulations containing different stabilizing additives (none, vitamin E, sucrose, and a combination of sucrose and vitamin E) as described in detail in Example 5.

FIG. 2 is a plot demonstrating the cumulative percentage of a model protein, bovine serum albumin, released from both poly(orthoester) and PLGA microspheres over time (measured at 1, 7, 14, 21, 27, and 37 days) in vitro in phosphate buffered saline at 37° C. as described in Example 7.

FIG. 3 is a plot demonstrating the in vitro degradation of bovine serum albumin contained in both poly(orthoester) microspheres and in PLGA microspheres at various time points (1, 7, 14, 21, 27, and 37 days) at 37° C. as described in Example 7.

FIG. 4 is a plot demonstrating the cumulative percentage of bovine serum albumin released from both poly(orthoester) and PLGA microspheres at various time points (1, 7, 14, 21, 27, and 37 days) in vitro in phosphate buffered saline at 50° C. as described in Example 7.

FIG. 5 is a plot is a plot demonstrating the in vitro degradation of bovine serum albumin contained in poly(orthoester) and in PLGA microspheres at various time points (1, 7, 14, 21, 27, and 37 days) at 50° C. as described in Example 7.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All publications, patents and patent applications cited herein, whether supra or infra, are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document which are incorporated herein by reference.

### Definitions

It must be noted that, as used in this specification, the singular forms “a,” “an,” and “the” include plural referents unless the context clearly dictates otherwise. Thus, for example, reference to a “polymer” includes a single polymer as well as two or more of the same or different polymers.

Unless specifically noted otherwise, definitions of the terms herein are standard definitions used in the arts of organic synthesis, and polymer and pharmaceutical science.

In describing and claiming the present invention, the following terminology will be used in accordance with the definitions described below.

A “biocompatible polymer” is a polymer having degradation products that are compatible with living tissue, or that may have beneficial biological properties. The biocompatible polymer may be biocompatible in itself, and/or may be synergistically biocompatible when employed in conjunction with a biologically active agent.

The term “reactive” refers to a functional group (e.g., present in a polymer) that reacts readily or at a practical rate under conventional conditions of organic synthesis. This is in contrast to those groups that either do not react or require strong catalysts or impractical reaction conditions in order to react (i.e., a “nonreactive” or “inert” group).

“Controlled release,” “sustained release,” “extended release” and similar terms are used to denote a mode of active agent (e.g., protein) delivery that occurs when an active agent is released from a delivery vehicle at an ascertainable and controllable rate over a period of time, rather than dispersed immediately upon administration. Controlled or sustained release may extend for hours, days or months, and may vary as a function of numerous factors.

“Molecular mass” in the context of a polyorthoester, refers to the nominal average molecular mass of a polymer, typically determined by size exclusion chromatography, light scattering techniques, or velocity. Molecular weight can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 1.2, less than about 1.15, less than about 1.10, less than about 1.05, and less than about 1.03.

The term, “delivery vehicle” refers to a composition that includes one or more of the following functions: the ability to transport an active agent to a site of interest, to control or affect the rate of access to, or release of, the active agent by sequestration or other means, and facilitate the provision of an active agent to a region in the body where its activity is needed.

The term, “matrix” denotes the physical structure of a polymer-based composition (e.g., a poly(ortho ester) which essentially retains an active agent such a protein in a manner which, in general, prevents or slows the release of the agent until the polymer erodes or decomposes.

“Semi-solid” refers to the mechano-physical state of a material that is flowable under moderate stress. More specifically, a semi-solid polymer will, in general, possess a viscosity between about 10,000 centipoise (cp) and 3,000,000 cp, especially between about 1,000,000 cp and 3,000,000 cp when measured neat at 25° C. Semi-solids generally have a Tg lower than room temperature. Preferably a formulation comprising a semi-solid polymer is syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

In contrast, a “solid” formulation, in reference to a poly(ortho ester) formulation as provided herein, is one that is a solid at room temperature. The solid, in general, is typically not flowable without the application of stress. Solid formulations generally have a Tg higher than room temperature.

The term, “room temperature” as used herein means 25° C.

“Treating” or “treatment” of a condition includes preventing the condition from occurring in a mammalian subject that may be predisposed to the condition but does not yet experience or exhibit symptoms of the condition (prophylactic treatment), inhibiting the condition (slowing or arresting its development), providing relief from the symptoms or side-effects of the condition (including palliative treatment), and relieving the condition (causing regression of the condition).

The term “drug,” or “pharmaceutically active agent” or “bioactive agent,” or “active agent” as used interchangeably, means any organic or inorganic compound or substance having bioactivity and adapted or used for therapeutic purposes. Proteins, hormones, anti-cancer agents, small molecule chemical compounds and mimetics, oligonucleotides, DNA, RNA and gene therapies are included under the broader definition of “drug”. As used herein, reference to a drug, as well as reference to other chemical compounds herein, is meant to include the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable.

“Protein” as used herein refers to a molecule containing 5 or more amino acid residues joined by peptide bonds. The term, protein, as used herein, is meant to encompass peptides, polypeptides and proteins, including antibodies and chimeric or fusion proteins, where such proteins may be modified by glycosylation, lipid attachment, sulfation, gamma-carboxylation of glutamic acid residues, hydroxylation, and the like.

The term “degradation” means a loss of protein integrity and/or bio-activity and/or structure.

“Glass transition temperature” or Tg, refers to the temperature at which an amorphous or partly amorphous material (in the case of polymers) undergoes a phase change from a hard, solid amorphous or partly amorphous material to a soft, rubbery liquid.

“Pharmaceutically acceptable excipient” or “pharmaceutically acceptable carrier” refers to an excipient (i.e., non-therapeutic agent) that can be included in the compositions provided herein and that causes no significant adverse toxicological effects to the patient.

The terms “effective amount” or “pharmaceutically effective amount” or “therapeutically effective amount” of a composition as provided herein (e.g., one containing one or more therapeutic proteins), refer to a non-toxic but sufficient amount of the composition to provide the desired response in an animal subject when administered. The exact amount required will vary from subject to subject, depending on the species, age, and general condition of the subject, the severity of the condition being treated, the particular active agent or agents employed, specifics of the composition, mode of administration, and the like. An appropriate “effective” amount in any individual case may be determined by one of ordinary skill in the art.

“Optional” or “optionally” means that the subsequently described circumstance may or may not occur, so that the description includes instances where the circumstance occurs and instances where it does not.

The term “substantially” in reference to a certain feature or entity means to a significant degree or nearly completely (i.e. to a degree of 85% or greater) in reference to the feature or entity.

The term “about”, particularly in reference to a given quantity, is meant to encompass deviations of plus or minus five percent.

Additional definitions may also be found in the sections which follow.

### Overview

The present disclosure is based, at least in part, on the provision of polymeric systems for extended delivery of proteins. In particular, provided herein are poly(orthoester)-based formulations that are effective for the sustained delivery of one or more therapeutic proteins. The formulations additionally maintain the stability and bioactivity (i.e., significantly minimize the degradation and/or aggregation) of the protein contained in the poly(orthoester) matrix during preparation, storage and release. In arriving at the subject matter disclosed herein, the inventors have recognized that a combination of certain factors is effective to minimize the degradation of proteins contained within a polymeric matrix, while also providing extended release of the protein over time. The formulations provided herein utilize poly(orthoesters) that have been tailored to minimize the formation of acidic products resulting from degradation of the poly(orthoester) matrix. The combined features of minimized polymer-derived acidic degradation products, coupled with the surface erosion property of the poly(orthoester) matrix, contribute to significantly reduce the formation of polymer-derived acidic degradation products trapped within the polymer matrix, to thereby provide an environment well-suited to preserve protein bioactivity.

In general, the formulations provided herein comprise a poly(orthoester) polymer combined with a therapeutic protein, where the poly(ortho ester) comprises less than 5 mole percent of α-hydroxy acid-containing subunits, and has a glass transition temperature (Tg), of greater than about −10° C.

The sustained release poly(orthoester)-based protein delivery systems, compositions, and related methods and uses will now be discussed in greater detail below.

### Methods of Preparing Poly(Orthoester)-Based Formulations

The poly(orthoesters) utilized in the instant disclosure are preferably solids at room temperature. This feature, i.e., that of a solid polymeric carrier, has also been discovered to contribute to the enhanced stability of a therapeutic protein contained therein. See, e.g., Examples 8-10. The poly(orthoester) utilized in the formulation will typically possess a glass transition temperature (Tg) of greater than about −10° C. or more preferably of greater than 0° C. Generally, poly(othoesters) suitable for use in the instant formulations and delivery systems described herein will generally possess a Tg of greater than about −10° C., or greater than about 0° C., or greater than about 10° C., or greater than about 15° C., or even greater than about 20° C. The glass transition temperature of a poly(orthoester) formulation can readily be determined by differential scanning calorimetry (DSC).

Additionally, the poly(orthoester) component comprises less than 5 mole percent of α-hydroxy acid-containing subunits, i.e., subunits derived from an α-hydroxy acid or a cyclic diester thereof, such as subunits comprising glycolide, lactide, or combinations thereof, i.e., poly(lactide-co-glycolide), including all ratios of lactide to glycolide, e.g., 75:25, 65:35, 50:50, etc. Such subunits are also referred to herein as latent acid subunits.

Generally, a poly(orthoester) for use in the compositions and delivery systems provided herein is described by the following formula:

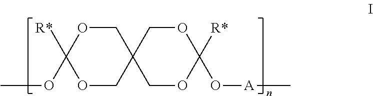

where:

R* is a C₁₋₄ alkyl (e.g., C1, C2, C3 or C4 alkyl); n is an integer ranging from 5 to 400, and A in each subunit is R¹, R², or R³, or preferably, is R¹ or R³. In a preferred embodiment, R* is ethyl.

In reference to structure I, R¹ is:

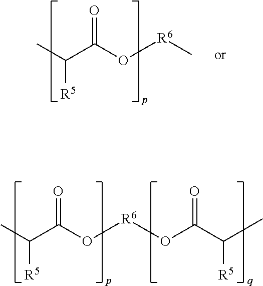

where p and q are each independently integers that range from between about 1 to 20 (e.g., are each independently selected from 1, 2, 3, 4, 5, 6, 7, 8, 9, 10, 11, 12, 13, 14, 15, 16, 17, 18, 19, and 20), each R⁵ is independently hydrogen or C_{1 -4} alkyl (e.g., is H, or C1, C2, C3, or C4 alkyl); and R⁶ is:

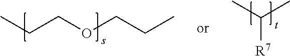

where s is an integer from 0 to 30 (e.g., is selected from , 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30) ; t is an integer from 2 to 200; and R⁷ is hydrogen or C₁ alkyl (e.g., is H or C1, C2, C3, or C4 alkyl). The R¹ subunits are α-hydroxy acid-containing subunits, i.e., subunits derived from an α-hydroxy acid or a cyclic diester thereof (also sometimes referred to as latent acids). As noted previously, the poly(orthoester) contains less than 5 mole percent of α-hydroxy acid containing subunits, to thereby provide an environment that is conducive to maintaining the integrity of a therapeutic protein entrapped therein.

R² is

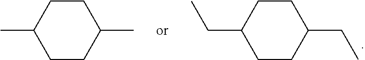

R³ is:

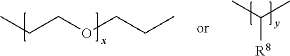

where x is an integer of 0 to 100, and is, in certain instances, preferably selected from 0, 1, 2, 3, 4, and 5; y is an integer of 2 to 40; and R⁸ is hydrogen or C₁₋₄ alkyl (C1, C2, C3 or C4 alkyl).

Preferably, the poly(orthoester) is one in which A is R¹ or R³, where R¹ is

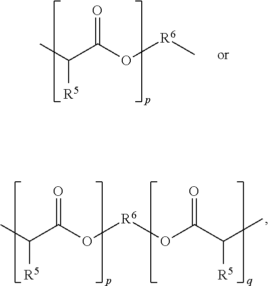

where p and q are each independently integers that range from between about 1 and 20, where the average number of p or the average number of the sum of p and q (p+q) is between about 1 and 7 (e.g., 1, 2, 3, 4, 5, 6, 7) in at least a portion of the monomeric units of the polymer; x and s are each independently an integer ranging from 0 to 30; and t and y are each independently an integer ranging from 2 to 40. In one or more preferred embodiments, R⁵ is H or methyl.

Exemplary polyorthoesters possess a molecular weight of about 2500 Da to about 200,000 Da, for example from 5,000 Da to about 150,000 Da or from 10,000 Da to 130,000 Da. Illustrative molecular weights, in Da, are 2500, 5000, 7500, 10,000, 20,000, 30,000, 40,000, 50,000, 60,000, 70,000, 80,000, 90,000, 100,000, 120,000, 150,000, 175,000 and 200,000, and ranges therein, wherein exemplary ranges include those formed by combining any one lower molecular weight as described above with any one higher molecular weight as provided above, relative to the selected lower molecular weight. Preferred poly(orthoesters) are solids at room temperature.

The poly(orthoester) can be prepared by reaction of a diketene acetal according to the formula:

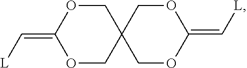

where L is hydrogen or a C₁₋₄ alkyl, with a diol according to formula HO—R¹—OH and at least one diol according to the formulae, HO—R²—OH, or HO—R³—OH (where R¹, R², R³ are as described above). In the presence of water, the α-hydroxy acid containing subunits are hydrolyzed at body temperature and at physiological pH to produce the corresponding hydroxy acids, which can then act as catalysts to control the hydrolysis rate of the polyorthoester without the addition of exogenous acid. The instant polyorthoesters have a lower degree of bioerodibility due to their low mole percentage of α-hydroxy acid containing subunits, thus lending to their suitability for extended delivery of proteins.

Preferred polyorthoesters are those in which the mole percentage of α-hydroxy acid containing subunits ranges from 0 to less than 5 mole percent. Exemplary percentages of α-hydroxy acid containing subunits in the poly(orthoester) polymer are from about 0.001 to about 4.99 mole percent, preferably from about 0.01 to about 4.5 mole percent, and from about 0.1 to about 4.0 mole percent. As an illustration, the percentage of α-hydroxy acid containing subunits may be 0.001, 0.01, 0.05, 0.10, 0.50, 1.0, 1.5, 2.0, 2.5, 3, 3.5, 4, 4.5, 4.8, or 4.9 mole percent, including any and all ranges lying therein, formed by combination of any one lower mole percentage number with any higher mole percentage number.

Additional preferred poly(orthoesters) are those in which A is R¹ or R³, where R¹ is

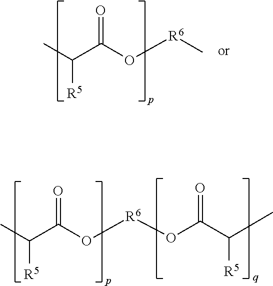

and p and q are each independently integers that vary from between about 1 and 20, or between about 1 and 15, or between about 1 and 10, where the average number of p or the average number of the sum of p and q (i.e., p+q) is between about 1 and 7 in at least a portion of the monomeric units of the polymer. Additionally, preferred ranges of x and s (in reference to the preferred embodiment above or in reference to any poly(orthoester as provided herein) are those in which each is independently an integer ranging from 0 to 30. Similarly, preferred ranges for t and y are those in which each independently varies from 2 to 40.

Preferred polyorthoesters are those in which R⁵ is hydrogen or methyl.

In yet a further embodiment, preferred are poly(orthoesters) that comprise less than about 15 mole percent of ethylene glycol-containing subunits such as

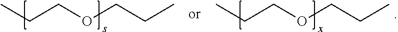

Limiting the amount of ethylene-glycol containing subunits in the poly(orthoester) has also been found to further contribute to the stability of the entrapped therapeutic protein. In certain preferred embodiments, s and x are each independently selected from 1, 2, 3, 4, 5, 6, 7 and 8. In one preferred embodiment, s is 3. In another preferred embodiment, x is 3.

An exemplary polyorthoester comprises alternating residues of 3,9-diethyl-3,9-2,4,8,10-tetraoxaspiro[5.5]undecane-3,9-diyl:

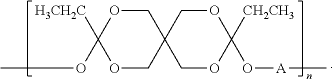

As an example, polyorthoesters such as those described above can be prepared by reacting an illustrative diketene acetal, 3,9-di(ethylidene)-2,4,8,10-tetraoxaspiro[5.5]undecane (DETOSU),

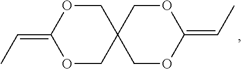

with one or more diols as described above, such as HO—R¹—OH, HO—R²—OH, or HO—R³—OH. Illustrative diols include ethylene glycols such as triethylene glycol (TEG), ethylene glycols modified at one or both termini with an α-hydroxy acid, organic diols having a hydrocarbyl core of from 2 to 40 carbon atoms such as 1,6-hexane diol, 1,10-decane diol, cis/trans 1,4-cyclohexane dimethanol, para-menthane-3,8-diol, 1,4-butane diol, 1,5-pentane diol, 1,7-heptane diol, 1,8-octane diol, and cyclic equivalents thereof, where the hydroxyl groups can be at any two positions within the cycloalkyl or alkylene ring.

Preferably, an organic diol will possess from 2 to 20 carbon atoms. The organic diol can be linear, branched or cyclic, and may also be saturated or unsaturated. Generally, unsaturated diols will possess from 1-3 elements of unsaturation. A preferred poly(orthoester) will contain from about from 10 to 50 total mole percent of subunits derived from one or more organic diols having a hydrocarbyl core, e.g., corresponding to R⁶ equal to

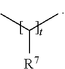

Diols such as HO—R¹—OH, HO—R²—OH, or HO—R³—OH are prepared as described in U.S. Pat. No. 5, 968,543 and in Heller et al., J. Polymer Sci., Polymer Letters Ed. 18:293-297 (1980). For example, a diol of the formula HO—R¹—OH comprising a polyester moiety can be prepared by reacting a diol of the formula HO—R³—OH with between 0.5 and 10 molar equivalents of a cyclic diester of an α-hydroxy acid such as lactide or glycolide, and allowing the reaction to proceed at 100-200° C. for about 12 hours to about 48 hours. Suitable solvents for the reaction include organic solvents such as dimethylacetamide, dimethyl sulfoxide, dimethylformamide, acetonitrile, pyrrolidone, tetrahydrofuran, and methylbutyl ether. Although the diol product is generally referred to herein as a discrete and simplified entity, e.g., TEG diglycolide (and diol reaction products such as TEG diglycolide), it will be understood by those of skill in the art that due to the reactive nature of the reactants, e.g., ring opening of the glycolide, the diol is actually a complex mixture resulting from the reaction, such that the term, TEG diglycolide, generally refers to the average or overall nature of the product.

A preferred polyorthoester is prepared by reacting 3,9-di(ethylidene)-2,4,8,10-tetraoxaspiro[5.5]undecane (DETOSU) with various reactive diols, see for example, Examples 1-4 and 6 herein. Generally, the polyorthoester is prepared by reacting DETOSU with two or more reactive diols as provided herein, and is carried out under anhydrous conditions.

Thus, in a particularly preferred embodiment, the polyorthoester is prepared by reacting at least (i) from 30 to 60 mole percent 3,9-di(ethylidene)-2,4,8,10-tetraoxaspiro[5.5]undecane, (ii) from 10 to 50 mole total mole percent of two or more organic diols having a hydrocarbyl core of from 2 to 40 carbon atoms, and optionally having 1 to 3 elements of unsaturation, and (iii) less than 5 mole percent of an a-hydroxy-acid containing polymeric reactant under conditions effective to provide a poly(orthoester) polymer having a Tg of greater than 0° C. and that is a solid at room temperature.

### Proteins and Peptides

The compositions and delivery systems provided herein comprise at least one therapeutic protein. Representative therapeutic proteins include proteins with enzymatic or regulatory activity (e.g., proteins for treating endocrine disorders or hormone deficiencies, proteins for haemostatis and thrombosis, proteins for treating metabolic enzyme deficiencies, proteins for treating pulmonary and gastrointestinal tract disorders, proteins for treating immunodeficiencies, proteins for haematopoiesis, proteins to treat infertility, proteins for immunoregulation, proteins for growth regulation), proteins for targeting (proteins for cancer treatment, immunoregulation), and proteins vaccines.

Examples of therapeutic proteins for inclusion in the poly(orthoester)-based delivery systems and compositions provided herein include insulin, pramlintide, growth hormone, insulin-like growth factor I, blood factor VIII, blood factor IX, antithrombin III, protein C, β-glucocerobrosidase, aglucosidase, laronidase, idursulphase, galsulphase, agalsidase-β, α-1 proteinase inhibitor, lactase, adenosine deaminase, human albumin, erythropoietin, darbepoetin, granulocyte colony stimulating factor, granulocyte macrophage colony stimulating factor, interleukin 11, follicle stimulating hormone, human chorionic gonadotropin, lutropin, interferons (interferon-α1, interferon α2a, interferon α2b, interferon αn3, interferon β1a, interferon β1b, interferonγ1b), interleukin2, tissue plasminogen activator, urokinase, Factor VII, salmon calcitonin, parathyroid hormone, octreotide, recombinant human bone morphogenic protein 2, recombinant human bone morphogenic protein 7, gonadotropin releasing hormone, keratinocyte growth factor, platelet-derived growth factor, a vascular endothelial growth factor trap protein, trypsin, nesiritide, collagenase, human deoxy-ribonuclease I, hyaluronidase, papain, asparaginase, rasburicase, lepuridin, bivalirudin, streptokinase, anisoylated plasminogen streptokinase activator complex, bevacizumab, cetuximab, panitumumab, alemtuzumab, rituximab, trastuzumab, abatacept, anakinra, adalimumab, etanercept, infliximab, alefacept, efalizumab, natalizumab, eculizumab, antithymocyte globulin (rabbit), basiliximab, daclizumab, muromonab-CD3, omalizumab, palivizumab, enfuvirtide, abciximab, ranibizumab, denileukin diftitox, ibritumomab tiuxetan, gemtuzumab ozogamicin, tositumomab, hepatitis B surface antigen, OspA, glucagon, growth hormone releasing hormone, secretin, thyroid stimulating hormone thyrotropin, among others.

Preferred proteins include those suitable for ophthalmic uses. Proteins belonging to this class include aflibercept and ranibizumab.

The amount of therapeutic protein contained within the instant formulations will vary depending upon the various components of the poly(orthoester), the particular protein employed, its bioactivity and molecular weight, and intended use, as well as the intended patient population, among other factors. In terms of percent by weight in the final formulation, a therapeutic protein will typically be loaded in amounts ranging from about 0.2% to up to about 75% by weight. Illustrative loading ranges for proteins are from about 0.5% to about 20% by weight (low loading), or from about 10% by weight to about 75% by weight (high loading). Thus, the formulations provided herein will typically contain one or more of the following weight percentages of protein: For example, the amount of therapeutic protein in the composition may vary from about 1 wt % to 75 wt %, 2 wt % to 70 wt %, 2 wt % to 60 wt %, 2 wt % to 50 wt %, or 3 wt % to 45 wt %, 5 wt % to 50 wt %, 10 wt % to 50 wt %, 10 wt % to 35 wt %, 10 wt % to 25 wt %, 1 wt % to 30 wt %, or 1 wt % to 25 wt %, and may be about 1 wt %, 5 wt %, 7 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, or up to 75 wt %. Suitable amounts, i.e., weight percentages, of therapeutic protein are those falling within any two of the representative weight percentages provided above.

Typically, proteins for use in the instant formulations are in dry form. The protein can be dried by any suitable drying method, for example, lyophilization, spray-drying, or supercritical drying. In some instances, the protein may be dried in the presence of one or more stabilizers, which may then be incorporated into the final formulation.

### Additives

The formulations provided herein may also contain one or more excipients or additives such as diluents, buffers, binders, thickeners, lubricants, preservatives (including antioxidants such as vitamin E as exemplified in Example 3), stabilizers, and surfactants (e.g., polysorbates such as “TWEEN 20” and “TWEEN 80”, and pluronics such as F68 and F88, available from BASF). For example, the formulation may comprise one or more carbohydrates, e.g., sugars or sugar alcohols. Suitable sugars include reducing and non-reducing sugars. Illustrative sugars include sucrose, trehalose, mannitol, sorbitol, sorbose, melezitose, raffinose, and the like. See, e.g., Example 3, in which sucrose was included in the formulation.

Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), and in “Handbook of Pharmaceutical Excipients”, 6th Ed., Ed. A. H. Kibbe, Pharmaceutical Press, 2009.

Generally, the formulations provided herein comprise from zero weight percent to up to about 75 weight percent excipient(s), depending upon the nature of the excipient. That is to say, the instant formulations may contain no excipients, or from about 1 weight % to 75 weight % excipient(s), or from about 5 weight % to 75 weight % excipient(s), or from about 10 weight percent to 65 weight percent excipient(s), or from about 15 weight percent to 50 weight percent excipient(s), etc. Illustrative weight percentages of one or combined excipients in the formulation are 1 wt %, 5 wt %, 7 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, or up to 75 wt %. Suitable amounts, i.e., weight percentages, of excipients are those falling within any two of the representative weight percentages provided above.

### Method of Preparation

The poly(orthoester)-based-therapeutic protein compositions are typically prepared by first mixing or blending the polyorthoester components as described above under anhydrous conditions to provide the poly(orthoester) component. The mixing or blending is performed by any suitable method, generally at a temperature less than about 50° C., e.g., at room temperature, although in certain instances, depending upon the nature of the materials, mixing or blending may be carried out at higher temperatures, e.g., from about 25 to 100° C., optionally in the presence of a solvent. Preferably, the poly(orthoester) product is a solid at room temperature.

The therapeutic protein, poly(orthoester), and any optional excipients and/or solvents are then combined to provide the desired extended release formulation. In certain preferred embodiments, the poly(orthoester)-therapeutic protein formulation is in the form of microparticles or rods.

Microparticles are typically prepared using preparing a solid-in-oil suspension method. In this approach for making microparticles, solid protein is suspended in an organic phase containing the poly(orthoester) and any other excipients. Solvents for use in the organic phase include dichloromethane, chloroform, tetrahydrofuran, ethyl acetate, or any other suitable organic solvent. Generally, a new solvent is then added to the mixture to form a new emulsion, e.g., a non-solvent for the poly(orthoester). Suitable non-solvents include water, with an emulsifier such as polyvinyl alcohol, or silicone oil, and optional additional additives such as sodium chloride. The resulting mixture is then stirred, resulting in the formation of microparticles.

Generally, during and/or after the emulsification step, the organic solvent is extracted or allowed to evaporate, resulting in the precipitation of microparticles comprising the therapeutic protein entrapped in the poly(orthoester) matrix. See, for example, Examples 1,2, 3, and 4, which describe the preparation of exemplary microparticles.

Alternatively, poly(orthoester)-based microparticles having a therapeutic protein entrapped therein can be prepared by spray drying, spray-freeze drying or supercritical fluid-based precipitation.

Generally, to prepare poly(orthoester)-based microparticles having a therapeutic protein entrapped therein by spray drying, a protein-poly(orthoester) dispersion as described above is sprayed through a heated nozzle, and the organic solvent is quickly evaporated by the hot gas flow. In a variation of the foregoing, microparticles may also be prepared using a cryogenic method in which a protein-poly(orthoester) dispersion is ultrasonically sprayed into liquid nitrogen over solid ethanol. During evaporation of the liquid nitrogen, melting of the ethanol occurs, resulting in extraction of the organic solvent from the microparticles formed during the spray drying process.

Alternatively, the microparticles may also be formed by precipitation using a supercritical fluid, usually carbon dioxide. In this methodology, a mixture of the therapeutic protein suspended in an organic poly(orthoester) solution is sprayed in supercritical carbon dioxide, which then results in dissolution of the organic solvent in the supercritical phase and precipitation of the protein-loaded poly(orthoester) microparticles.

After formation, the resultant microparticles are typically washed, e.g., with distilled water, and dried to remove any residual solvent, e.g., by air-drying, lyophilization, or vacuum-drying.

The resulting poly(orthoester) microparticles will generally have a range of diameters from about 10 microns to about 500 microns. Illustrative microparticles sizes will range from about 10-500 microns, from about 10-400 microns, from about 10-300 microns, from about 10 to 200 microns, or from about 10-100 microns, or from 4 microns to 80 microns. For ophthalmic applications, a preferred size range is from about 20-100 microns.

The resulting microparticles may be in the shape of spheres, e.g., microspheres, or may also be provided in different shapes such as discs and rods. Microparticles having different shapes such as discs or rods can be prepared by electrohydrodynamic co-jetting of the poly(orthoester) polymer solutions, by varying the solution and process parameters, such as concentration and flow rate (Bhaskar, S., et al., Small, 6 (3), 2010, 404-411).

### Features

The poly(orthoester)-based delivery systems provided herein are effective to provide extended release of a therapeutic protein entrapped therein. Moreover, the instant formulations are designed to promote stability of entrapped protein by minimizing protein degradation and/or aggregation. The instant formulations provide an optimal combination and balance of physical state, as provided by the preferred glass transition temperatures of the poly(orthoester) to provide a solid poly(orthoester) at room temperature, minimal exposure of the entrapped protein to an acidic environment within the poly(orthoester) matrix by virtue of the molar percentage of α-hydroxy acid-containing subunits (latent acid), and the like.

Formulations as provided herein are generally capable of providing sustained release of the therapeutic protein over a period of at least 3 days, or over a period of at least 5 days, preferably over a period of at least 7 days, or even more preferably over a period of at least 10 days. In certain instances, the formulation is capable of providing sustained release of the therapeutic protein for a period of at least 14 days, or even greater. In a particular embodiment, the poly(orthoester)-based delivery system is effective to provide sustained release of protein over a period of at least 10 days, or even over a period of at least 14 days, or over a period of at least 22 days, when evaluated in vitro in phosphate buffered saline at 37° C.

See, e.g., Example 5, which illustrates the release of the model protein, bovine IgG, from various poly(ortho ester) microsphere preparations. The various formulations were prepared using illustrative polyorthoester formulation AP141 (see Table 1). The illustrative poly(orthoester) was prepared from the following mole percentages of reactants: 4.88 mole percent triethylene glycol diglycolide/48.78 mole percent DETOSU/7.32 mole percent triethylene glcol (TEG)/and 39.02 mole percent cis and trans-1,4-cyclohexane dimethanol. Microspheres were prepared with IgG and poly(orthoester) only; IgG, poly(orthoester) and vitamin E; IgG, poly(orthoester) and sucrose, and IgG, poly(orthoester), vitamin E and sucrose.

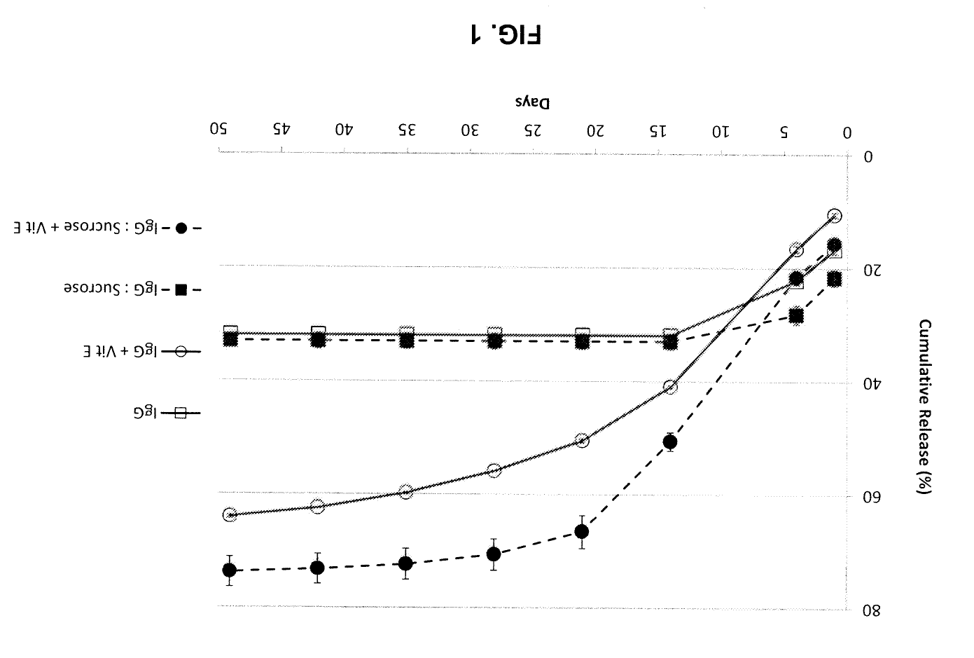

As can be seen from FIG. 1, the representative poly(orthoester) microsphere preparations are effective to provide extended release of antibody over time. The IgG and IgG sucrose-containing microspheres demonstrated a cumulative release of IgG of about 30% over time, with release of the antibody leveling off at about 14 days. Both vitamin E-containing microsphere formulations demonstrated extended release of IgG over a period of about 50 days. The IgG-vitamin E microsphere formulation released about 64% IgG over a period of about 50 days, while the IgG-vitamin E-sucrose formulation was effective to release about 74% IgG over the same period. Thus, the exemplary poly(ortho ester) formulation, AP141, containing 2.44 mole percent poly(D,L-lactic-co-glycolic) acid ester, 2.44 mole percent triethylene glycol mono-lactide, 7.32 mole percent triethylene glycol, 48.78 mole percent DETOSU and 39.02 mole percent cis and trans-1,4-cyclohexane dimethanol (relative molar ratios of 1:1:3:16) was effective to provide sustained release of the exemplary antibody, IgG, over time. The incorporation of one or more additives such as vitamin E and/or sucrose was effective to further modify/tailor the cumulative release of antibody over time, demonstrating the versatility of the instant microsphere formulations.

In this example, the addition of vitamin E to the poly(ortho ester) microsphere formulation was effective to enhance the cumulative release of antibody three-fold (when compared to the IgG and IgG-sucrose formulations), while the further addition of sucrose to the vitamin E-containing formulation was effective to further enhance the cumulative release of antibody over time. The exemplary poly(ortho ester) formulation contains a significant molar excess of hydrophobic polymer subunits relative to the TEG, TEG-LA and PLGA ester-containing subunits. This feature (among others), i.e., an excess of hydrophobic polymeric subunits, has been found to confer advantages to the resulting formulations, such as enhanced stability of the therapeutic proteins contained therein.

The instant formulations are also effective to stabilize the therapeutic protein, e.g., upon formulation, during storage, and upon release. Preferably, an entrapped protein will exhibit no more than about 25% degradation (as measured by intact monomeric or native form of the protein, or no more than 25% loss in bioactivity. Even more preferably, an entrapped protein will exhibit no more than about 20% degradation, or no more than about 20% loss in bioactivity. Even more preferably, an entrapped protein will exhibit no more than about 15% degradation, or no more than about 15% loss in bioactivity, or no more than about 10% degradation, or no more than about 10% loss in bioactivity. The stability of the entrapped protein may be measured immediately following formulation, or at various time points during storage. Formulations such as those provided herein are typically stored under anhydrous conditions at ambient temperature and pressure, typically in a sealed pouch, and preferably under an inert atmosphere, e.g., for 1 month, or for 3 months, or for 6 months. Alternatively, the stability of a formulation can be measured under in vitro conditions to simulate the in-vivo environment, e.g., in vitro in phosphate buffered saline at 37° C., at various time points, e.g., at day 1, at day 2, at day 3, at day 4, at day 5, at day 6, at day 7, and so forth. Levels of protein can be measured by techniques such as HPLC and ELISA; three dimensional conformation of proteins may be analyzed using size-exclusion chromatography-HPLC, SDS-PAGE, circular dichroism or fourier transform infrared spectroscopy or other methods as appropriate. In one embodiment, a composition as provided herein is characterized by a degree of degradation of the therapeutic protein of no more than 25% when evaluated in vitro in phosphate buffered saline at 37° C. at day 7.

Further to this point, Example 6 describes the preparation of various poly(orthoester) formulations as described in Tables 1 and 2. The poly(orthoesters) were prepared using various molar amounts of three of more of the following: triethylene glycol-latent acid, in this case, triethylene glycol glycolide, DETOSU, triethylene glycol, 1,6-hexane diol, 1,10-decane diol, and cis and trans-1,4-cyclohexane dimethanol. The model protein, bovine serum albumin, was then added to the poly(orthoesters) to prepare exemplary poly(orthoester)-based films.

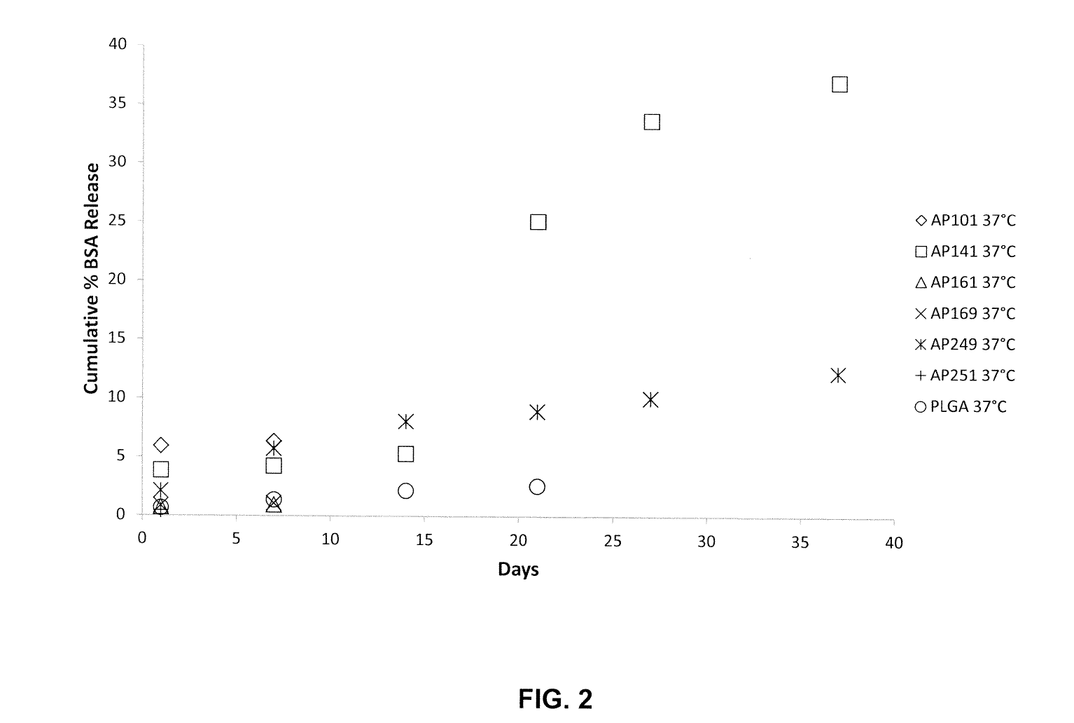

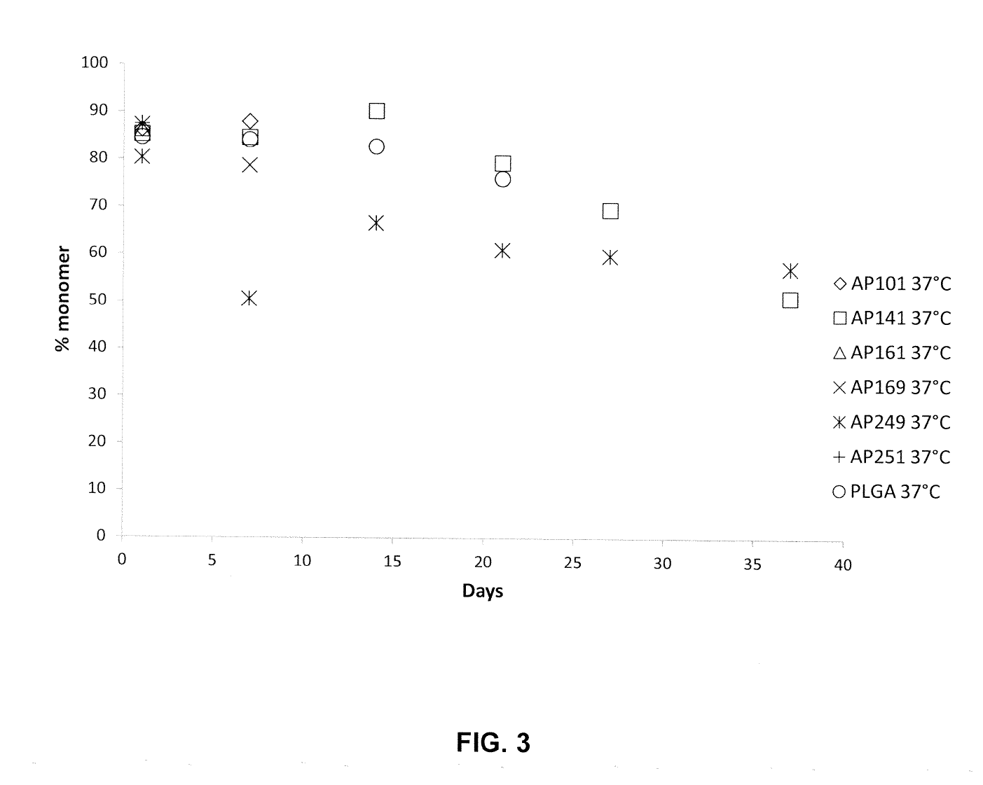

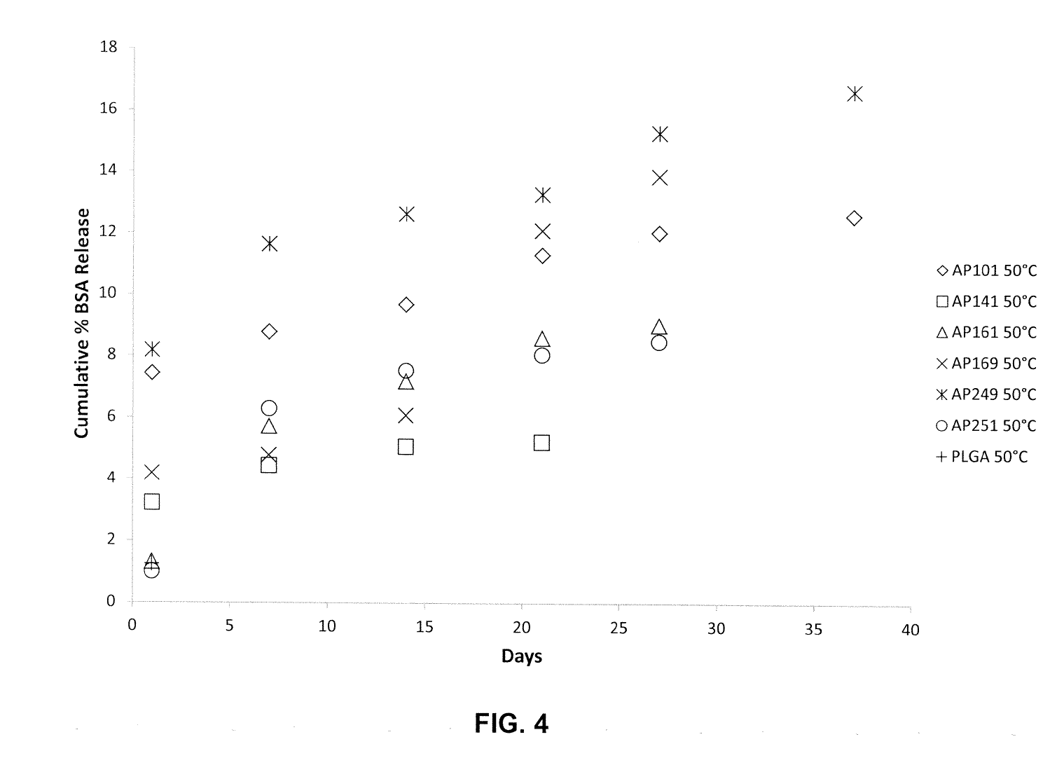

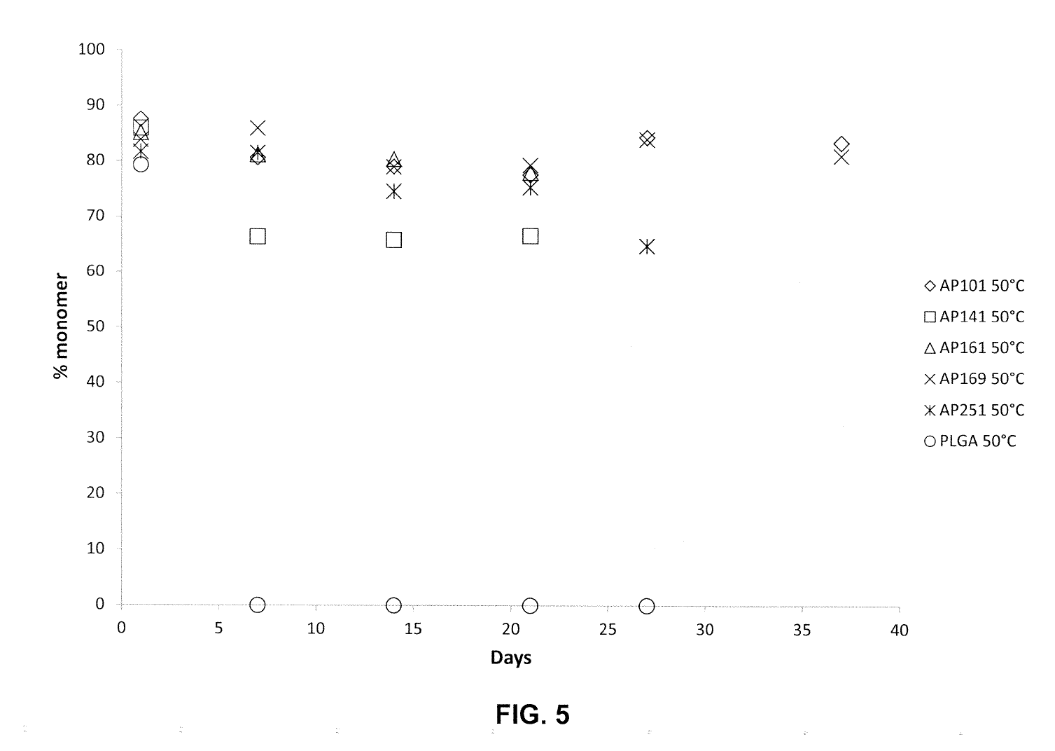

Results indicating the cumulative percentage of bovine serum albumin released from both poly(orthoester) and PLGA microspheres over time in vitro in phosphate buffered saline at 37° C. and at 50° C. are provided in FIGS. 2 and 4, respectively. FIGS. 3 and 5 are plots demonstrating the extent of in vitro degradation of bovine serum albumin contained in these microspheres at various time points measured at 37° C. and at 50° C., respectively. Most striking is the data in Table 3 which illustrates the vast different in stability of the model protein in the instant poly(orthoester) formulations in comparison to a PLGA-based formulation when measured at 50° C. By day 7, essentially all of the bovine serum albumin had degraded in the PLGA-based formulation, while protein stability in the poly(orthoester) formulations was retained, on average, from about 88% to about 81%, with one formulation, AP141, characterized by 66% intact protein. These examples demonstrate that formulations in accordance with this disclosure provide a notable improvement over PLGA microspheres both in maintaining the integrity/stability (i.e., preventing degradation) of entrapped protein, as well as providing improved efficiency of release of protein from the carrier systems provided herein.

### Method of Administration

The therapeutic-protein based formulations provided herein are useful for treating any condition that is treatable by administration of the therapeutic protein. That is to say, the formulations provided herein are administered to a subject in need thereof, e.g., a mammalian subject such as a human, in a therapeutically effective amount, for treating a condition that is treatable by administration of the therapeutic protein. Generally, the formulations provided herein are administered parenterally, by intravenous, intramuscular, intra-arterial, or by subcutaneous injection, or at the site of a tumor. Microparticulate-based formulations are typically dispersed in a pharmacologically-acceptable aqueous medium, optionally containing one or more dispersing agents or isotonic agents. The dosage amount and dosing regimen are determined based upon the particular therapeutic protein, the particular poly(orthoester) polymer components, the condition to be treated, route of administration, age and body weight of the patient, severity of the condition to be treated, and the like. In general, a dosage amount of 0.10 milligrams to 100 milligrams of therapeutic protein, preferably 1 milligram to 10 milligrams of therapeutic protein is administered in each dosage form, where, due to the extended delivery profile provided by the instant formulations, exemplary dosing regimens are preferably once a week, or twice a week, or three times a week, or once every two weeks, or once every three weeks.

The present application will now be described in connection with certain embodiments and examples which are not intended to limit the scope of the disclosure. On the contrary, the present application covers all alternatives, modifications, and equivalents as included within the scope of the claims. Thus, the following will illustrate the practice of the present application, for the purposes of illustration of certain embodiments and is presented to provide what is believed to be a useful and readily understood description of its procedures and conceptual aspects.

### EXAMPLES

### Abbreviations

c,t-CDM: mixture of cis and trans-1,4-cyclohexane dimethanol

PLGA: poly(D,L-lactic-co-glycolic) acid, lactide:glycolide 50:50, ester terminated, 7-17 kDa

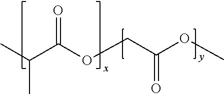

DETOSU: 3,9-di(ethylidene)-2,4,8,10-tetraoxaspiro[5.5]undecane,

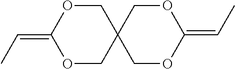

TEG: triethylene glycol

TEG-LA: triethylene glycol-latent acid

### Materials

Poly(ortho esters) were prepared by reacting 3,9-di(ethylidene)-2,4,8,10-tetraoxaspiro[5.5]undecane (DETOSU),

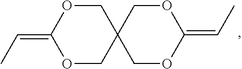

with one or more of the following as set forth in Table 1 below: triethylene glycol (TEG), triethylene glycol-glycolide (generally referred to asTEG-LA), 1,6-hexane diol, 1,10-decane diol, and/or cis and trans-1,4-cyclohexane dimethanol, in tetrahydrofuran under rigorous anhydrous conditions as described generally Heller, J., et al., Biomacromolecules 2004, 5 (5), 1625-32 and in U.S. Pat. No. 5,968,543.

The mole percentages of each of the components of the illustrative poly(orthoester) formulations are provided in the table below.

TABLE 1
Illustrative Poly(ortho ester) Formulations:
Components - Mole Percentages

| | | | | 1,6- | 1,10- | |
|---|---|---|---|---|---|---|
| | TEG- | | | hexane | decane | c,t- |
| Designation | Glycolide | DETOSU | TEG | diol | diol | CDM |
| AP101 | | 49.5 | | | 30.3 | 20.2 |
| AP141 | 4.88 | 48.78 | 7.32 | | | 39.02 |
| AP161 | 0.10 | 49.5 | 5.05 | | 20.1 | 25.2 |
| AP169 | 0.10 | 50.5 | | 9.85 | | 39.6 |
| AP249 | | 49.75 | | 25.13 | 25.13 | |
| AP251 | | 49.75 | | 50.25 | | |
| AP91 | 0.50 | 49.37 | 5.04 | | 24.94 | 20.15 |
| AP93 | 2.50 | 48.88 | 4.99 | | 23.69 | 19.95 |
| AP95 | 0.10 | 49.47 | | | 30.24 | 20.19 |
| AP97 | 1.00 | 49.25 | | | 29.65 | 20.1 |
| AP98 | 2.50 | 48.88 | | | 28.68 | 19.95 |
| AP119 | 1.00 | 49.6 | | | 29.4 | 20 |

TABLE 2
Properties of Various Poly(ortho ester) Compositions

| | Lot | Mw | | |
|---|---|---|---|---|
| Designation | number | (kDa) | Solid | Tg (° C.)* |
| AP101 | 993091 | 20 | yes | n/a |
| | | | | variable |
| AP141 | 1010117 | 94 | yes | n/a ~70 |
| AP161 | 1149131 | 44 | yes | n/a ~40 |
| AP169 | 84194 | 120 | | n/a ~85 |
| | | | yes | |
| AP249 | | | yes | n/a |
| AP251 | | | yes | n/a |
| AP91 | 72910 | | yes | |
| AP93 | n/a | | yes | 27 |
| AP95 | 72642 | | yes | n/a ~30 |
| AP97 | 72644 | | yes | n/a ~28 |
| AP98 | 72645 | | yes | n/a ~31 |
| AP119 | 68138 | | yes | |

*Tg values were not available for the specific lots (not available for any lots of AP249 or AP251); the values provided are an approximation based upon other lots of the same poly(ortho ester) composition.
Tg values were determined by differential scanning calorimetry.

### Example 1

### Preparation of Poly(Orthoester) Microspheres Containing Albumin

Bovine serum albumin was ground using a mixer mill (MM301, Retsch). 50 mg of this ground material was suspended in 0.5 mL of dichloromethane (DCM). The suspension was transferred to a glass vial containing 500 mg of poly(ortho ester) AP141 dissolved in 0.5 mL of dichloromethane. The two samples were mixed by vortexing. The resulting solid-in-oil suspension was homogenized in 5 mL of 4% polyvinyl alcohol solution at 11500 rpm for 1 min (T10 homogenizer, IKA). The resulting solid/oil/water emulsion was poured into 45 mL of 4% polyvinyl alcohol and stirred at 20° C. for 3 h to allow for solvent evaporation. The hardened microspheres were filtered, washed with distilled water and lyophilized.

### Example 2

### Preparation of Poly(Ortho Ester) Microspheres Containing Albumin with Vitamin E Acetate as an Additive

Bovine serum albumin was ground using a mixer mill (MM301, Retsch). 50 mg of the ground material was combined with 50 mg of vitamin E acetate and suspended in 0.5 mL of dichloromethane. The suspension was transferred to a glass vial containing 500 mg of poly(ortho ester) AP141 dissolved in 0.5 mL of dichloromethane. The two samples were mixed by vortexing. The resulting solid-in-oil suspension was homogenized in 5 mL of 4% polyvinyl alcohol solution at 11500 rpm for 1 min (T10, IKA). The formed solid/oil/water emulsion was poured into 45 mL of 4% polyvinyl alcohol and stirred at 20° C. for 3 h to allow for solvent evaporation. The hardened microspheres were filtered, washed with distilled water and lyophilized.

### Example 3

### Preparation of Poly(Ortho Ester) Microspheres Containing an Antibody

For the IgG formulation absent additional stabilizers, 500 mg of lyophilized bovine IgG was ground using a mixer mill (MM301, Retsch). 100 mg of this ground material was suspended in 0.5 mL of dichloromethane. This suspension was transferred to a glass vial containing 500 mg of poly(ortho ester) AP141 dissolved in 0.5 mL of dichloromethane. The two samples were mixed by vortexing. This solid-in-oil suspension was homogenized in 5 mL of 4% polyvinyl alcohol solution with 5 w/v % sodium chloride at 11500 rpm for 1 min (T10, IKA). The resulting solid/oil/water emulsion was poured into 45 mL of 4% polyvinyl alcohol containing 5 w/v % sodium chloride and stirred at 20° C. for 3 h to allow for solvent evaporation. The hardened microspheres were filtered, washed with distilled water and lyophilized

An additional sucrose-containing formulation was prepared as follows. An aqueous solution containing 500 mg of bovine IgG and 500 mg of sucrose was lyophilized and then ground using a mixer mill (MM301, Retsch). 100 mg of this ground material was suspended in 0.5 mL of dichloromethane. This suspension was transferred to a glass vial containing 500 mg of poly(ortho ester) AP141 dissolved in 0.5 mL of dichloromethane. The two samples were mixed by vortexing. This solid-in-oil suspension was homogenized in 5 mL of 4% polyvinyl alcohol solution with 5 w/v % sodium chloride at 11500 rpm for 1 min (T10, IKA). The resulting solid/oil/water emulsion was poured into 45 mL of 4% polyvinyl alcohol containing 5 w/v % sodium chloride and stirred at 20° C. for 3 h to allow for solvent evaporation. The hardened microspheres were filtered, washed with distilled water and lyophilized.

### Example 4

### Preparation of Poly(Ortho Ester) Microspheres Containing Antibody with Vitamin E Acetate as Additive

An aqueous solution containing 500 mg of bovine IgG and 500 mg of sucrose was lyophilized and then ground using a mixer mill (MM301, Retsch). 100 mg of this ground material was combined with 50 mg vitamin E acetate and suspended in 0.5 mL of dichloromethane. This suspension was transferred to a glass vial containing 500 mg of poly(ortho ester) AP141 dissolved in 0.5 mL of dichloromethane. The two samples were mixed by vortexing. This solid-in-oil suspension was homogenized in 5 mL of 4% polyvinyl alcohol solution with 5 w/v % sodium chloride at 11500 rpm for 1 min (T10, IKA). The resulting solid/oil/water emulsion was poured into 45 mL of 4% polyvinyl alcohol containing 5 w/v % sodium chloride and stirred at 20° C. for 3 h to allow for solvent evaporation. The hardened microspheres were filtered, washed with distilled water and lyophilized.

### Example 5

### In Vitro Release of Antibody from Poly(Orthoester) Microspheres

Thirty milligrams of various poly(ortho ester) microsphere preparations containing bovine IgG were weighed into low protein binding eppendorf tubes. To each of these was added 1 mL of phosphate buffered saline at pH 7.4 containing 0.02 w/v % sodium azide. Samples were incubated at 37° C. At predetermined time points, the samples were centrifuged and supernatant was removed and replaced with an equal volume of fresh buffer. The concentration of IgG released was quantified by HPLC. An Agilent Bio SEC-3 column, 4.6×300 mm, 3 μm particle size and 300 μm pore size was used with phosphate buffered saline at pH 7.4 containing 0.02 w/v % sodium azide as the mobile phase at a flowrate of 0.4 mL/min. The percentage of antibody released was then calculated from a standard curve of known concentrations of bovine IgG. This was performed in duplicate. See FIG. 1.

As can be seen from FIG. 1, the poly(ortho ester) microsphere preparations are effective to provide extended release of antibody over time. While the IgG and IgG sucrose-containing microspheres demonstrated a cumulative release of IgG of about 30% over time, with release of the antibody leveling off at about 14 days, after which time essentially no more antibody was released, both vitamin E-containing microsphere formulations demonstrated extended release of IgG over a period of about 50 days. The IgG-vitamin E microsphere formulation released about 64% IgG over a period of about 50 days, while the IgG-vitamin E-sucrose formulation was effective to release about 74% IgG over the same period. Thus, the exemplary poly(ortho ester) formulation, AP141, containing 2.44 mole percent poly(D,L-lactic-co-glycolic) acid ester, 2.44 mole percent triethylene glycol mono-lactide, 7.32 mole percent triethylene glycol, 48.78 mole percent DETOSU and 39.02 mole percent cis and trans-1,4-cyclohexane dimethanol (relative molar ratios of 1:1:3:16) was effective to provide sustained release of the exemplary antibody, IgG, over time. The incorporation of one or more additives such as vitamin E and/or sucrose is effective to further modify/tailor the cumulative release of antibody over time, demonstrating the versatility of the instant microsphere formulations. In this example, the addition of vitamin E to the poly(ortho ester) microsphere formulation was effective to enhance the cumulative release of antibody three-fold (when compared to the IgG and IgG-sucrose formulations), while the further addition of sucrose to the vitamin E-containing formulation was effective to further enhance the cumulative release of antibody over time. The exemplary poly(ortho ester) formulation contains a significant molar excess of hydrophobic polymer subunits relative to the TEG, TEG-LA and PLGA ester-containing subunits. This feature (among others), i.e., an excess of hydrophobic polymeric subunits, has been found to confer advantages to the resulting formulations, such as enhanced stability of the therapeutic proteins contained therein.

### Example 6

### Preparation of Poly(Ortho Ester) and Poly(D,L-lactic-co-glycolic) Acid Films Containing Albumin

Each of various poly(ortho esters), AP101, AP141, AP161, AP169, AP249, and AP251, and a poly(D,L-lactic-co-glycolic) acid (lactide:glycolide 50:50, ester terminated, 7-17 kDa) at 200 mg each were dissolved in 200 μL of dichloromethane. To each of the resulting solutions was added 20 mg of bovine serum albumin suspended in dichloromethane. The resulting liquid compositions were mixed by vortexing, cast onto glass slides and allowed to dry overnight.

### Example 7

### Albumin Degradation in Poly(Ortho Ester) Systems and in Poly(D,L-lactic-co-glycolic) Acid System

Thirty milligrams of the polymer/protein mixtures prepared in Example 6 were weighed into low protein binding eppendorf tubes. To each polymer/protein mixture was added 1 mL of phosphate buffered saline at pH 7.4 containing 0.02 w/v % sodium azide. As controls, bovine serum albumin at 10 mg/ml was prepared in each of pH 2, 5 and 7.4 phosphate buffered saline. In vitro release was carried out at 37° C. and 50° C. At predetermined time points, the samples were centrifuged and supernatant was removed and replaced with an equal volume of fresh buffer.

The concentration of albumin released and the monomer percentage were quantified by HPLC. An Agilent Bio SEC-3 column, 4.6×300 mm, 3 μm particle size and 300 μm pore size was used with phosphate buffered saline at pH 7.4 containing 0.02 w/v % sodium azide as the mobile phase at a flow rate of 0.4 mL/min. The percentage of albumin released was then calculated from a standard curve of known concentrations of bovine serum albumin. The percentage of albumin in the monomer form was calculated based on the total bovine serum albumin released. The above was performed in duplicate. See FIGS. 2, 3, 4, and 5.

FIG. 2 and FIG. 4. demonstrate the cumulative release of BSA at various time points at 37° C. and 50° C. respectively, while FIG. 3 and FIG. 5. demonstrate the stability of the protein as indicated by percent monomer at 37° C. and 50° C. respectively. As illustrated in the plots, the PLGA-based microspheres fail to release significant amounts of protein and fail to maintain sustained released with about 3% of protein released at 37° C. (stopped releasing after 21 days) and 1% of protein released at 50° C. (stopped releasing after 1 day). The extent of degradation of protein was greatest in the PLGA-microsphere formulation. The non-liquid (i.e., solid) poly(ortho ester) formulations exhibited higher cumulative release of protein and also maintained protein stability to a greater extent than the PLGA formulation. At 50° C., as expected, the cumulative release of protein was on average higher, with the various formulations exhibiting release of BSA over time (FIG. 4). As shown in FIG. 5, the complete degradation of protein was observed for the PLGA-based formulation at day 7, while the poly(ortho ester) formulations maintained good protein stability up to at least day 27 as illustrated in Table 3 below. Based upon the table below, it can be seen that the poly(orthoester) formulations provided are effective to maintain the stability of proteins incorporated therein.

TABLE 3
Degradation of Albumin at 50° C.

Percent Monomer
Day

| Formulation | 1 | 7 | 14 | 21 | 27 | 37 |
|---|---|---|---|---|---|---|
| AP101 | 88 | 81 | 79 | 78 | 84 | 83 |
| AP141 | 86 | 66 | 66 | 67 | — | — |
| AP161 | 85 | 81 | 80 | 78 | 73 | — |
| AP169 | 89 | 88 | 80 | 84 | 83 | — |
| AP249 | 84 | 86 | 79 | 79 | 84 | 81 |
| AP251 | 82 | 81 | 75 | 75 | 65 | — |
| PLGA | 79 | — | — | — | — | — |

— = no albumin signal detected.

As can be seen from the figures, formulations in accordance with this disclosure provide a notable improvement over PLGA microspheres both in maintaining the integrity/stability (i.e., preventing degradation) of entrapped protein, as well as providing improved efficiency of release of protein from the microsphere carriers.

### Example 8

### Formulation of a Peptide in a Semi-Solid Poly(Orthoester) Having a Tg Less than 0° C.

A poly(orthoester) designated as AP240 was prepared using the following relative molar amounts of reactants: 0.10 mole % TEG glycolide, 47.37 mole % DETOSU and 52.58 mole % TEG as described previously The poly(orthoester), while possessing 0.10 mole percent (i.e., a low amount) of glycolide-containing subunits, and having a Tg of less than −10° C., is a semi-solid at room temperature.

### Example 9

### Preparation of A GLP-1 Poly(Orthoester) Formulation

17.8 mg of GLP-1 (7-37) was weighed into a vial with an aqueous solution of 0.8 mg of base. The resulting GLP-1 solution was then lyophilized. To the lyophilized GLP-1 was added 317 mg of a poly(orthoester) compatible excipient, N-methyl pyrrolidone, and the resulting mixture was thoroughly mixed until homogenous. A semi-solid poly(orthoester) polymer, AP240, as described in Example 8, containing 0.1 mole % triethylene glycol diglycolide and having a Tg of less than −10° C., was heated to 80° C., and then 743 mg of the heated poly(orthoester) was added to the mixture of N-methyl pyrrolidone and GLP-1. The formulation was mixed thoroughly and analyzed by HPLC.

### Example 10

### In-Vitro Release of GLP-1 Formulated with a Semi-Solid Poly(Orthoester)

A formulation as described in Example 9 was weighed into vials and filled with a phosphate buffered saline solution. The vials were then stored at 37° C. and analyzed by HPLC to monitor for the release of GLP-1 (7-37) from the formulation. The in-vitro release of the GLP-1 averaged 16% within the first 24 hours, and then proceeded to a cumulative release total of about 30% after 48 hours. After about 168 hours, only 38% of the GLP-1 (7-37) had been released from the formulation. Release of GLP-1 from the formulation essentially ceased at a cumulative release of 45% reached somewhere between about 168 and 360 hours. No monomeric GLP-1 was detected after the 360 hour time point, and aggregates were visually observed in the phosphate buffered saline in vitro release buffer.

### Example 11

### Preparation of a Copaxone (Glatiramer Acetate) Poly(Orthoester) Formulation

139 mg of glatiramer acetate (formerly known as copolymer-1) was added to a vial containing a homogenous mixture of 751 mg of poly(orthoester) AP240, as described in Example 8, and 309 mg of N-methyl pyrrolidone. The poly(orthoester), AP240, was heated to 80° C. prior to transfer to the vial. The resulting suspension was mixed thoroughly and analyzed by HPLC.

### Example 12

### In-Vitro Release of Glatiramer Acetate Formulated with a Semi-Solid Poly(Orthoester)

The formulation described in Example 11 was weighed into vials, followed by addition of phosphate buffered saline. The vials were then stored at 37° C. and analyzed by HPLC to monitor for the release of glatiramer acetate from the formulation. Cumulative in vitro release of the glatiramer acetate averaged about 10% within the first 24 hours. After about 48 hours, the cumulative release was only at about 37%. After 11 days (264 hours), about 62% of the glatiramer acetate had been released from the formulation. Maximal release of glatiramer acetate from the formulation was observed at 360 hours, by which time 70% of therapeutic protein had been released.

1. A composition effective to provide extended release of a protein, the composition comprising a poly(orthoester) combined with a therapeutic protein, wherein the poly(orthoester) comprises less than 5 mole percent of α-hydroxy acid-containing subunits, and has a glass transition temperature (Tg) of greater than about −10° C.

2. The composition of claim 1, having a Tg of greater than about 0° C.

3. The composition of claim 1, having a Tg of greater than about 10° C.

4. The composition of 1, wherein the poly(orthoester) comprises from 0.01 to 4.9 mole percent α-hydroxy acid-containing subunits selected from subunits comprising lactide, glycolide or combinations thereof.

5. The composition of claim 1, wherein the poly(orthoester) comprises zero mole percent α-hydroxy acid-containing subunits.

6. The composition of claim 1, wherein the poly(orthoester) has the structure: 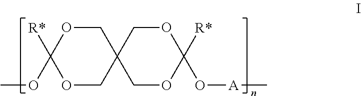
where:
R* is a C₁₋₄ alkyl;
n is an integer ranging from 5-400;
A in each subunit is R¹, R², or R³, where
R¹ is: 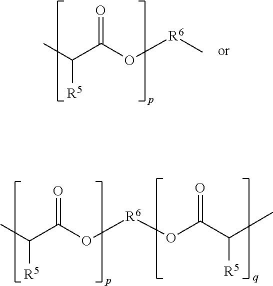
where:
p and q are each independently integers that vary from between about 1 to 20,
each R⁵ is independently hydrogen or C₁₋₄ alkyl; and
R⁶ is: 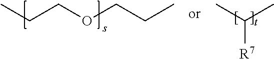
where s is an integer from 0 to 30;
t is an integer from 2 to 200; and
R⁷ is hydrogen or C₁₋₄ alkyl;
R² is: 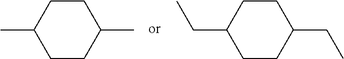
R³ is: 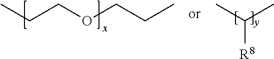
where:
x is an integer of 0 to 100;
y is an integer of 2 to 40; and
R⁸ is hydrogen or C₁₋₄ alkyl.

7. The composition of claim 6, wherein A is R¹ or R³, where R¹ is 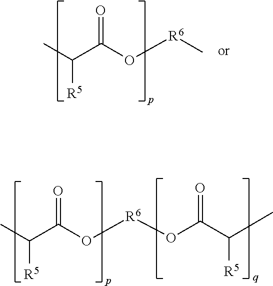
where p and q are each independently integers that vary from between about 1 and 20, where the average number of p or the average number of the sum of p and q (p+q) is between about 1 and 7 in at least a portion of the monomeric units of the polymer;
x and s are each independently an integer ranging from 0 to 30; and
t and y are each independently an integer ranging from 2 to 40.

8. The composition of claim 7, where R⁵ is H or methyl.

9. The composition of claim 6, where x is selected from 0, 1, 2, 3, 4, and 5.

10. The composition of claim 1, wherein the therapeutic protein is selected from the group consisting of insulin, pramlintide, growth hormone, insulin-like growth factor I, blood factor VIII, blood factor IX, antithrombin III, protein C, β-glucocerobrosidase, aglucosidase, laronidase, idursulphase, galsulphase, agalsidase-β, α-1 proteinase inhibitor, lactase, adenosine deaminase, human albumin, erythropoietin, darbepoetin, granulocyte colony stimulating factor, granulocyte macrophage colony stimulating factor, interleukin 11, follicle stimulating hormone, human chorionic gonadotropin, lutropin, interferons (interferon-α1, interferon α2a, interferon α2b, interferon αn3, interferon β1a, interferon β1b, interferonγ1b), interleukin2, tissue plasminogen activator, urokinase, Factor VII, salmon calcitonin, parathyroid hormone, octreotide, recombinant human bone morphogenic protein 2, recombinant human bone morphogenic protein 7, gonadotropin releasing hormone, keratinocyte growth factor, platelet-derived growth factor, a vascular endothelial growth factor trap protein, trypsin, nesiritide, collagenase, human deoxy-ribonuclease I, hyaluronidase, papain, asparaginase, rasburicase, lepuridin, bivalirudin, streptokinase, anisoylated plasminogen streptokinase activator complex, bevacizumab, cetuximab, panitumumab, alemtuzumab, rituximab, trastuzumab, abatacept, anakinra, adalimumab, etanercept, infliximab, alefacept, efalizumab, natalizumab, eculizumab, antithymocyte globulin (rabbit), basiliximab, daclizumab, muromonab-CD3, omalizumab, palivizumab, enfuvirtide, abciximab, ranibizumab, denileukin diftitox, ibritumomab tiuxetan, gemtuzumab ozogamicin, tositumomab, hepatitis B surface antigen, OspA, glucagon, growth hormone releasing hormone, secretin, thyroid stimulating hormone thyrotropin.

11. The composition of claim 6, wherein the poly(orthoester) is a solid at room temperature.

12. The composition of clam 11 in the form of microparticles or cylindrical rods.

13. The composition of claim 6, effective to provide sustained release of the therapeutic protein over a period of at least 10 days when evaluated in vitro in phosphate buffered saline at 37° C.

14. The composition of claim 13, further characterized by a degree of degradation of the therapeutic protein of no more than 25% when evaluated in vitro in phosphate buffered saline at 37° C. at day 7.

15. The composition of claim 6, wherein the combined mole percentage of 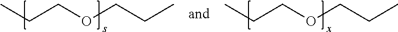
in the poly(orthoester) is less than 15.

16. The composition of claim 1, where the poly(orthoester) is prepared by reacting at least (i) from 30 to 60 mole percent 3,9-di(ethylidene)-2,4,8,10-tetraoxaspiro[5.5]undecane, (ii) from 10 to 50 mole total mole percent of two or more organic diols having a hydrocarbyl core of from 2 to 40 carbon atoms, and optionally having 1 to 3 elements of unsaturation, and (iii) less than 5 mole percent of an α-hydroxy-acid containing polymeric reactant under conditions effective to provide a poly(orthoester) polymer having a Tg of greater than 0° C. and that is a solid at room temperature.

17. The composition of claim 16, wherein the two or more organic diols have a hydrocarbyl core of from 2 to 20 carbon atoms.

18. The composition of claim 16, further comprising, in the reacting step, a hydroxyl or α-hydroxy-acid terminated ethylene-glycol with from 2 to 30 subunits.

19. The composition of claim 18, wherein the hydroxyl or α-hydroxy-acid terminated ethylene glycol is a triethylene glycol.

20. A plurality of microparticles comprising a solid therapeutic protein contained within a poly(orthoester) matrix, wherein the poly(ortho ester) comprises less than 5 mole percent of α-hydroxy acid-containing subunits, and has a glass transition temperature (Tg), of greater than about −10° C.

21. The plurality of microparticles of claim 20, comprising the poly(orthoester) of claim 6.

22. The plurality of microparticles of claim 21, having sizes ranging from about 4 microns to about 80 microns.

23. The plurality of microparticles of claim 22, wherein the microparticles are microspheres.

24. A plurality of microparticles of claim 21, where the plurality of microparticles is effective to provide extended release of the therapeutic protein over a period of time that is extended by at least two-fold when compared to the release of the same therapeutic protein from a plurality of PLGA (50:50) microspheres when evaluated in vitro in phosphate buffered saline at 37° C. at day 7.

25. The plurality of microspheres of claim 24, wherein the therapeutic protein is characterized by a degree of degradation of no more than 25% when evaluated in vitro in phosphate buffered saline at 37° C. at day 7.

26. A method for enhancing the stability of a therapeutic protein upon encapsulation in a polymeric matrix by combining the therapeutic protein in a poly(orthoester) matrix, wherein the poly(orthoester) comprises less than 5 mole percent of α-hydroxy acid-containing subunits, and has a glass transition temperature (Tg), of greater than about −10° C.

27. The method of claim 26, wherein the poly(orthoester) matrix is as recited in claim 6.

28. The method of claim 27, wherein the method further comprises providing extended release of the therapeutic protein from the polymeric matrix over a period of at least 7 days when evaluated in in vitro in phosphate buffered saline at 37° C.

29. A method of treating a mammalian subject for a condition that is treatable by administration of a therapeutic protein, the method comprising administering to the subject a therapeutically effective amount of the a composition comprising a poly(orthoester) combined with a therapeutic protein, wherein the poly(orthoester) comprises less than 5 mole percent of α-hydroxy acid-containing subunits, and has a glass transition temperature (Tg) of greater than about −10° C.

30. A method of treating a mammalian subject for a condition that is treatable by administration of a therapeutic protein, the method comprising administering to the subject a therapeutically effective amount of a plurality of microparticles comprising a solid therapeutic protein contained within a poly(orthoester) matrix, wherein the poly(ortho ester) comprises less than 5 mole percent of α-hydroxy acid-containing subunits, and has a glass transition temperature (Tg) of greater than about −10° C.
